(19) United States
(12) Patent Application Publication
(10) Pub. No.: US 2019/0357754 A1
(43) Pub. Date: Nov. 28, 2019

(54) MEDICAL SYSTEM

(71) Applicant: OLYMPUS CORPORATION, Tokyo (JP)

(72) Inventor: Kosuke MOTAI, Saitama (JP)

(73) Assignee: OLYMPUS CORPORATION, Tokyo (JP)

(21) Appl. No.: 16/532,572

(22) Filed: Aug. 6, 2019

Related U.S. Application Data
(63) Continuation of application No. PCT/JP2017/017255, filed on May 2, 2017.

(51) Int. Cl. A61B 1/00; A61B 90/00

(52) U.S. Cl. CPC A61B 1/00137; A61B 2090/034; A61B 90/03; A61B 1/00135

(57) ABSTRACT

A medical system includes: an overtube having a first lumen that extends in a longitudinal direction toward a distal end from a proximal end, a second lumen that branches off from the distal end of the first lumen and opens at a distal-end surface, and that is in communication with the first lumen via a first entrance, and a third lumen that opens at an outer circumferential surface and that is in communication with the first lumen via a second entrance; and an attachment that is inserted, together with a medical device, into the first lumen from the proximal-end side, that has a lateral cross-sectional shape making it possible to selectively block at least a portion of either the first or second entrance as a result of the position thereof about a center axis of the first lumen being changed and to guide the medical device to the other entrance.

### CROSS-REFERENCE TO RELATED APPLICATIONS

This is a continuation of International Application PCT/JP2017/017255 which is hereby incorporated by reference herein in its entirety.

### TECHNICAL FIELD

The present invention relates to a medical system.

### BACKGROUND ART

There is a known medical system in which a raising base is disposed, in a pivotable manner, at a distal-end portion in a lumen that passes through an overtube in a longitudinal direction thereof, and a viewing-field direction of an imaging catheter introduced into the lumen is switched between a direct-viewing direction and a lateral-viewing direction by pivoting the raising base by pulling a wire attached to the raising base (for example, see Patent Literature 1).

### CITATION LIST

### Patent Literature

{PTL1} PCT International Publication No. WO 2007/063904

### SUMMARY OF INVENTION

An aspect of the present invention is a medical system including: an overtube that has a first lumen that extends in a longitudinal direction toward a distal end from a proximal end, a second lumen that branches off from the distal end of the first lumen and opens at a distal-end surface, and that is in communication with the first lumen via a first entrance, and a third lumen that opens at an outer circumferential surface and that is in communication with the first lumen via a second entrance; and an elongated attachment that is inserted, together with an elongated medical device, into the first lumen of the overtube from the proximal-end side, and that has a lateral cross-sectional shape that makes it possible to selectively blocks at least a portion of either the first entrance or the second entrance as a result of the position thereof about a center axis of the first lumen being changed and to guide the medical device to the other entrance.

### BRIEF DESCRIPTION OF DRAWINGS

FIG. 1 is an overall configuration diagram showing a medical system according to an embodiment of the present invention.

FIG. 2 is a longitudinal cross-sectional view showing a distal-end portion of an overtube provided in the medical system in FIG. 1.

FIG. 3 is a lateral cross-sectional view in which the overtube in FIG. 2 is viewed as facing the distal-end side.

FIG. 4 is a perspective view showing a proximal-end-side opening of a first lumen in the overtube in FIG. 2.

FIG. 5 is a longitudinal cross-sectional view showing a distal-end portion of an attachment provided in the medical system in FIG. 1.

FIG. 6 is a lateral cross-sectional view showing a state in which a medical device and the attachment are inserted into the first lumen of the overtube in FIG. 2 at a first phase.

FIG. 7 is a lateral cross-sectional view showing a state in which the medical device and the attachment are inserted into the first lumen of the overtube in FIG. 2 at a second phase.

FIG. 8 is a perspective view showing a state in which the attachment is inserted in the proximal-end-side opening of the first lumen in FIG. 4.

FIG. 9 is a longitudinal cross-sectional view showing a state in which the medical device and the attachment are inserted into the first lumen of the overtube in FIG. 2.

FIG. 10 is a longitudinal cross-sectional view showing a state in which a distal end of the attachment is abutted against an abutting surface of the overtube from the state in FIG. 9.

FIG. 11 is a longitudinal cross-sectional view showing a state in which the medical device is made to protrude forward from the overtube via a second lumen from the state in FIG. 10.

FIG. 12 is a longitudinal cross-sectional view showing a state in which the medical device is made to protrude sideward from the overtube via a third lumen.

FIG. 13 is a perspective view showing a modification of the attachment of the medical system in FIG. 1.

FIG. 14 is a perspective view showing a state in which two treatment tools and an observation apparatus are made to protrude from a distal-end surface of the overtube of the medical system in FIG. 1.

FIG. 15 is a perspective view showing a state in which the two treatment tools and the observation apparatus are made to protrude from an outer circumferential surface of the overtube of the medical system in FIG. 1.

### DESCRIPTION OF EMBODIMENT

A medical system 1 according to an embodiment of the present invention will be described below with reference to the drawings.

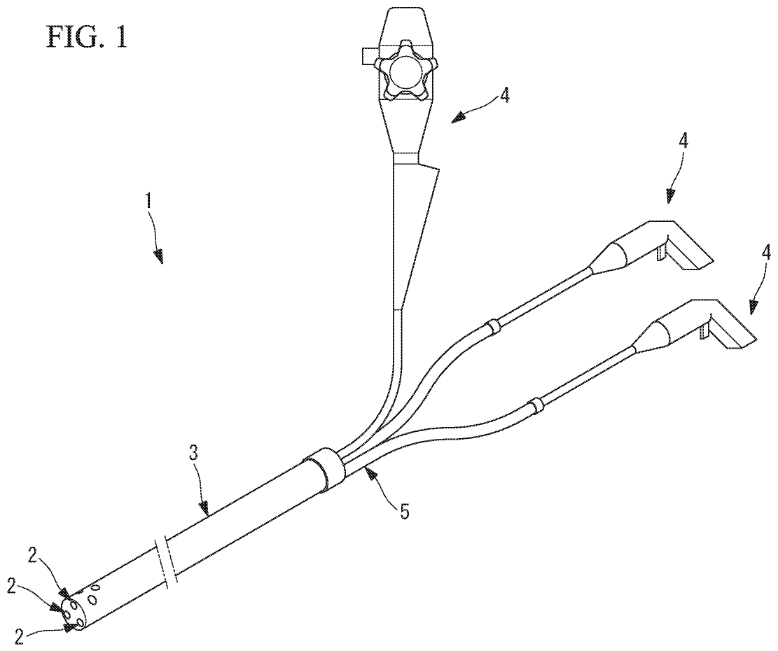

As shown in FIG. 1, the medical system 1 according to this embodiment includes: an elongated overtube 3 that possesses flexibility and that is provided with a lumen provided along a longitudinal direction thereof; a medical device 4 that is inserted into the lumen 2 of the overtube 3 from a proximal-end side thereof; and an attachment 5 that is inserted into the lumen 2 together with the medical device 4 from the proximal-end side thereof.

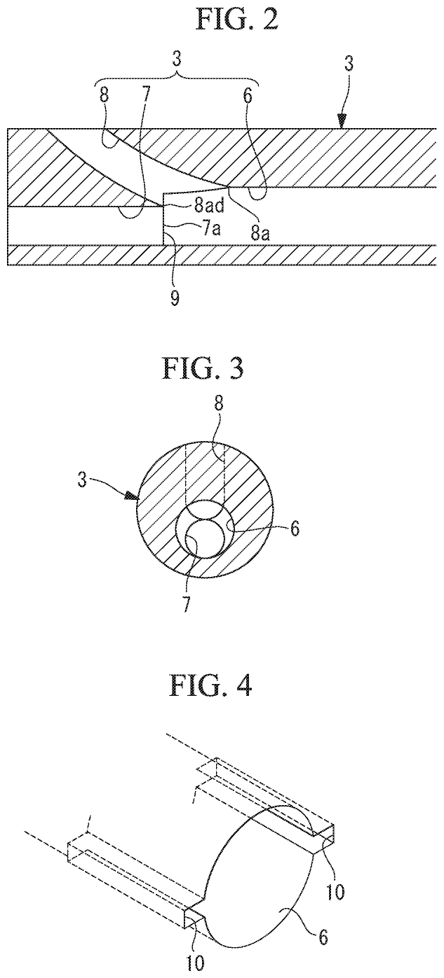

As shown in FIG. 2, the lumen 2 of the overtube 3 includes: a first lumen 6 that extends along the longitudinal direction to the vicinity of a distal-end portion from a proximal-end surface; a second lumen 7 that branches off from a distal end of the first lumen 6, that opens at a distal end of the overtube 3, and that is in communication with the first lumen 6 via an entrance (first entrance) 7a; and a third lumen 8 that branches off from the distal end of the first lumen 6, that opens at an outer circumferential surface of the overtube 3, and that is in communication with the first lumen 6 via an entrance (second entrance) 8a. In the figure, reference sign 8ad is a distal-end edge of the second entrance 8a.

One of or a plurality of the lumens 2 formed of the first lumen 6, the second lumen 7, and the third lumen 8 may be provided in the overtube 3. Although FIG. 1 shows a case in which a plurality of lumens 2 are provided in the overtube 3, this embodiment will be described in terms of an example in which a single lumen 2 is provided in order to simplify the description.

The first lumen 6, the second lumen 7, and the third lumen 8 each has a circular lateral cross-sectional shape. The second lumen 7 has an inner diameter that is smaller than the inner diameter of the first lumen 6, and has a large enough size to allow the insertion of the medical device 4. As shown in FIGS. 2 and 3, the second lumen 7 is eccentrically disposed with respect to the first lumen 6 so that an inner surface thereof comes into contact with an inner surface of the first lumen 6.

The third lumen 8 also has an inner diameter that is smaller than the inner diameter of the first lumen 6, and has a large enough size to allow the insertion of the medical device 4. As shown in FIG. 3, the third lumen 8 is disposed on the opposite side from the direction in which the second lumen 7 is eccentrically disposed, and, as shown in FIG. 2, the third lumen 8 gradually bend toward the distal end along a plane that includes the center axis of the first lumen 6, and opens at an outer circumferential surface of the overtube 3.

At a position at which the second lumen 7 and the third lumen 8 branch off from the first lumen 6, a step difference due to the difference in the inner diameters of the first lumen 6 and those of the second lumen 7 and the third lumen 8 is formed. This step difference forms an abutting surface 9 against which a distal end of the attachment 5 that has been inserted into the first lumen 6 is abutted. In addition, in the longitudinal direction, it is preferable that the abutting surface 9 be positioned in alignment with the distal-end edge 8ad of the second entrance 8a. In addition, it is preferable that the abutting surface 9 be positioned at the first entrance 7a in the longitudinal direction of the overtube 3.

As shown in FIG. 4, the overtube 3 is provided with, at two locations at 180°-intervals in a circumferential direction, slit-like grooves (slits) 10 that extend radially outward from the inner surface of the first lumen 6 along the longitudinal direction of the first lumen 6 from the opening at the proximal-end surface thereof. The groove widths and lengths of the grooves 10 are set to be large enough to allow protrusions 13 of the attachment 5, described later, to be accommodated therein.

The medical device 4 is, for example, an imaging catheter, an endoscope, or a treatment tool such as gripping forceps.

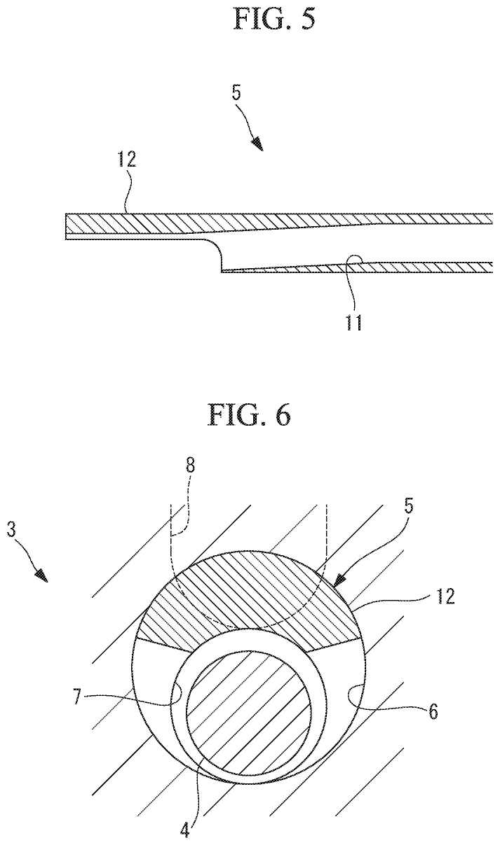

As shown in FIG. 5, the attachment 5 is formed in a tube-like shape that possesses flexibility and that includes an inner hole 11 into which the medical device 4 can be inserted. The external shape of the attachment 5 is formed in a slightly smaller size than the inner diameter of the first lumen 6, and the attachment 5 is inserted into the first lumen 6 in the longitudinal direction in a movable manner in a state in which the medical device 4 is inserted into the inner hole 11.

The attachment 5 includes a distal-end portion 12 that has an arc-like lateral cross-sectional shape as a result of a portion thereof in a circumferential direction being cut out over a predetermined length from the distal end thereof.

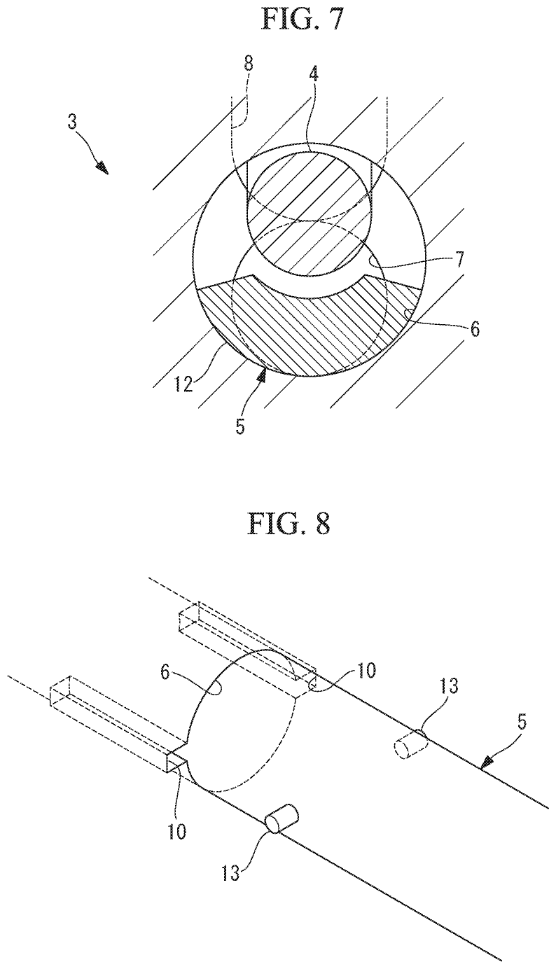

As shown in FIG. 5, the distal-end portion 12 is formed, for example, having a greater wall thickness as compared with other portions of the attachment 5, and, as shown in FIGS. 6 and 7, the distal-end portion 12 has a shape that nearly fills up a gap between the outer surface of the medical device 4 and the inner surface of the first lumen 6 on one side of the medical device 4 in a radial direction in the state in which the distal-end portion 12 is inserted into the first lumen 6 together with the medical device 4.

In addition, as shown in FIG. 8, the attachment 5 is provided with the two protrusions 13 that protrude radially outward from the outer circumferential surface at a position at which the attachment 5 is aligned with the proximal-end surface of the overtube 3 in a state in which the distal end of the attachment 5 is disposed at a position that is separated by a predetermined distance with respect to the abutting surface 9 provided at the branching position between the first lumen 6 and the second lumen 7. It is possible to move the attachment 5 forward in the first lumen 6 by moving the protrusions 13 in the grooves 10 in the longitudinal direction by rotating the attachment 5 about the center axis of the first lumen 6 so that one of the protrusions 13 of the attachment 5 is aligned with one of the grooves 10 provided in the first lumen 6 and so that the other protrusion 13 is aligned with the other groove 10, and by pressing the attachment 5 toward the distal end in that state.

Specifically, in this state, a state in which the phase of the attachment 5 about the center axis with respect to the first lumen 6 is fixed at a first phase is maintained. In this embodiment, the distal end of the attachment 5 is disposed on a third-lumen-8 side at this first phase so that the first entrance 7a to the second lumen 7 is opened, whereas at least a portion of the second entrance 8a to the third lumen 8 is blocked.

In addition, it is possible to move the attachment 5 forward in the first lumen 6 by moving the protrusions 13 in the grooves 10 in the longitudinal direction by rotating the attachment 5 about the center axis of the first lumen 6 so as to switch the positions of the two protrusions 13 with respect to the two grooves 10 and by pressing the attachment 5 toward the distal end in this state. By doing so, it is possible to maintain the phase of the attachment 5 about the center axis with respect to the first lumen 6 at a second phase.

In this embodiment, the distal end of the attachment 5 is disposed on a second-lumen-7 side at this second phase so that the second entrance 8a to the third lumen 8 is opened, whereas at least a portion of the first entrance 7a to the second lumen 7 is blocked.

Note that it is possible to appropriately select the combination of the protrusions 13 and the grooves 10 for achieving the first phase or the second phase, for example, by marking a surface of the attachment 5 exposed outside the body in a state in which the protrusions 13 have not been inserted into the grooves 10.

The operation of the medical system 1 according to this embodiment, thus configured, will be described below.

In order to treat an affected portion in a body cavity by using the medical system 1 according to this embodiment, the overtube 3 is inserted into the body cavity, and the medical device 4, to which the attachment 5 is attached, is inserted into the first lumen 6 from the proximal-end surface of the overtube 3 exposed outside the body.

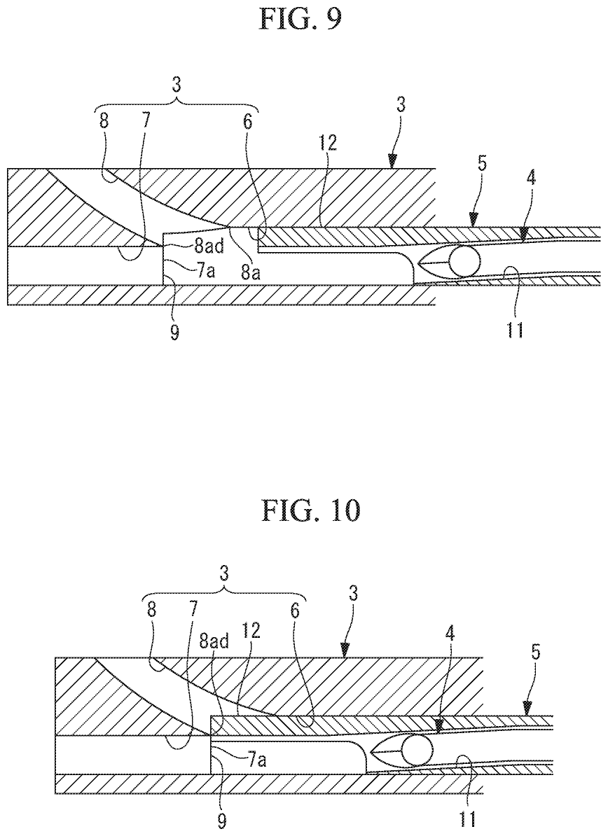

At this time, as shown in FIG. 9, the medical device 4 is inserted into the inner hole 11 of the attachment 5, and the medical device 4 is disposed in a state in which the distal end of the medical device 4 is retracted farther on the proximal-end side than the distal end of the attachment 5 is. Then, at a position at which the distal end of the attachment 5 is away from the abutting surface 9 by a predetermined distance before reaching the abutting surface 9, the attachment 5 is rotated about the center axis of the first lumen 6 so that the protrusions 13 of the attachment 5 in the portion thereof that is disposed outside the body is aligned with one of the grooves 10.

As a result of inserting the attachment 5 into the first lumen 6 in the state in which the protrusions 13 are positioned with respect to the grooves 10 so as to achieve the first phase of the attachment 5 with respect to the first lumen 6, the attachment 5 and the medical device 4 are moved forward in the first lumen 6 while the first phase is maintained due to the engagement between the grooves 10 and the protrusions 13 in the circumferential direction. Then, as shown in FIG. 10, when the distal end of the attachment 5 abuts against the abutting surface 9, the distal-end portion 12 of the attachment 5 blocks the second entrance 8a to the third lumen 8 and the first entrance 7a to the second lumen 7 is opened.

By doing so, the medical device 4 is easily guided into the second lumen 7, as shown in FIG. 11, by merely moving the medical device 4 forward in the longitudinal direction with respect to the attachment 5. Thus, by doing so, it is possible to observe or treat an affected portion that is positioned in front of the overtube 3 by making the medical device 4 protrude from the distal-end surface of the overtube 3.

On the other hand, as a result of inserting the attachment 5 into the first lumen 6 in the state in which the protrusions 13 are positioned with respect to the grooves 10 so as to achieve the second phase of the attachment 5 with respect to the first lumen 6, the attachment 5 and the medical device 4 are moved forward in the first lumen 6 while the second phase is maintained due to the engagement between the grooves 10 and the protrusions 13 in the circumferential direction. Then, when the distal end of the attachment 5 abuts against the abutting surface 9, the distal-end portion 12 of the attachment 5 blocks the first entrance 7a to the second lumen 7 and the second entrance 8a to the third lumen 8 is opened.

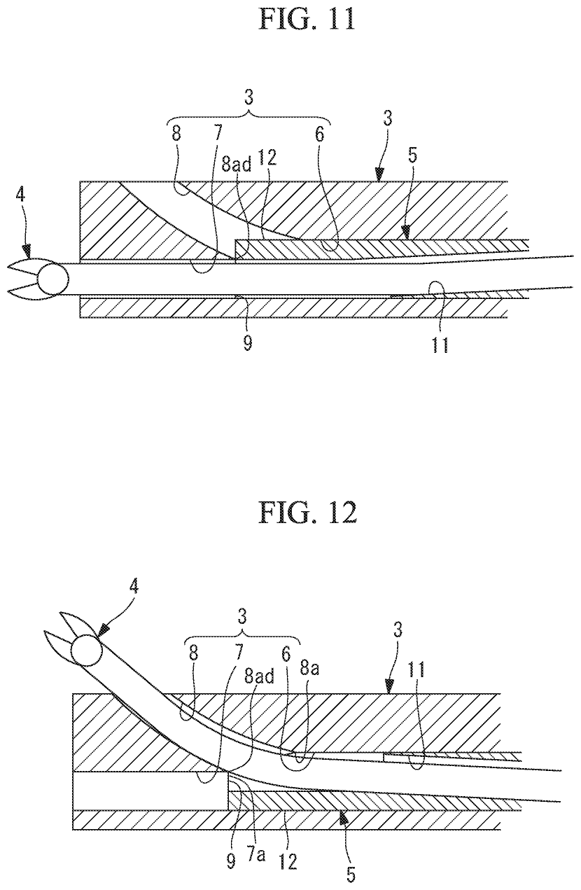

By doing so the medical device 4 is easily guided into the third lumen 8, as shown in FIG. 12, by merely moving the medical device 4 forward in the longitudinal direction with respect to the attachment 5. Thus, by doing so, it is possible to observe or treat an affected portion that is laterally positioned with respect to the overtube 3 by making the medical device 4 protrude from the outer circumferential surface of the overtube 3.

In addition, in the case in which an affected portion that is laterally positioned with respect to the overtube 3 needs to be observed or treated in the state in which the affected portion positioned in front of the overtube 3 is being observed or treated by making the medical device 4 protrude from the distal-end surface of the overtube 3, first, the medical device 4 is moved back with respect to the attachment 5, and the medical device 4 is disposed farther on the proximal-end side than the distal end of the attachment 5 is. Subsequently, the attachment 5 is moved back from the first lumen 6 toward the proximal end, thus releasing the engagement between the protrusions 13 and the grooves 10.

Then, the attachment 5 is disposed at the second phase by being rotated about the center axis of the first lumen 6 with respect to the overtube 3. By doing so, the position of the distal-end portion 12 of the attachment 5 in the first lumen 6 is switched due to the rotation of the attachment 5. In this state, as a result of moving the attachment 5 forward again in the state in which the protrusions 13 and the grooves 10 are engaged with each other and abutting the distal end thereof against the abutting surface 9, the first entrance 7a to the second lumen 7 is blocked and the second entrance 8a to the third lumen 8 is opened. Subsequently, the medical device 4 is easily guided into the third lumen 8 by moving the medical device 4 forward with respect to the attachment 5, and thus, it is possible to make the medical device 4 protrude from the outer circumferential surface of the overtube 3.

As has been described above, with the medical system 1 according to this embodiment, it is not necessary to provide a raising base and a wire for pivoting the raising base in the overtube 3, and it is possible to easily switch between forward and sideward protruding of the medical device 4 with respect to the overtube 3 by merely rotating the attachment 5 about the center axis of the first lumen 6. In other words, there is an advantage in that it is possible, with a simple structure, to selectively make the medical device 4 protrude either forward in the longitudinal direction of the overtube 3 or in the direction that intersects the longitudinal direction.

In addition, with this embodiment, by inserting the protrusions 13 of the attachment 5 into the grooves 10 provided in the inner surface of the first lumen 6, it is possible to maintain the attachment 5 so as not to rotate with respect to the first lumen 6 about the center axis thereof. By doing so, there is an advantage in that it is possible to maintain the two phases in which the medical device 4 is made to protrude forward and sideward with respect to the overtube 3 so that the respective phases are not changed.

Also, in order to switch between the first phase and the second phase, it suffices to move back the attachment 5 to the position at which the engagement between the protrusions 13 and the grooves 10 is released; therefore, it is not necessary to pull out, by a large amount, the medical device 4 and the attachment 5 from the first lumen 6, and thus, there is an advantage in that it is possible to change the direction in which observation or treatment is performed in a simple manner within a short period of time.

In addition, in this embodiment, because the distal end of the attachment 5 is inserted until the distal end abuts against the abutting surface 9, it is possible to reliably realize the state in which the medical device 4 is made to protrude forward or sideward with respect to the overtube 3 without requiring an operator to pay attention to the amount by which the attachment 5 is inserted. Note that, because it is also possible to achieve the state in which the medical device 4 is made to protrude forward or sideward with respect to the overtube 3, without providing the abutting surface 9, by inserting the attachment 5 to the vicinity of the position at which the second lumen 7 and the third lumen 8 branch off from the first lumen 6, the abutting surface 9 need not be provided.

In addition, in this embodiment, as the attachment 5, an example having a tube shape in which only the distal-end portion 12 is cut out has been described. Although it is preferable to employ a tube shape because doing so makes it possible to increase the torsional rigidity, an attachment 5 that has a certain arc-like lateral cross-sectional shape over the entire length thereof may be employed so long as it is possible to ensure an enough torsional rigidity.

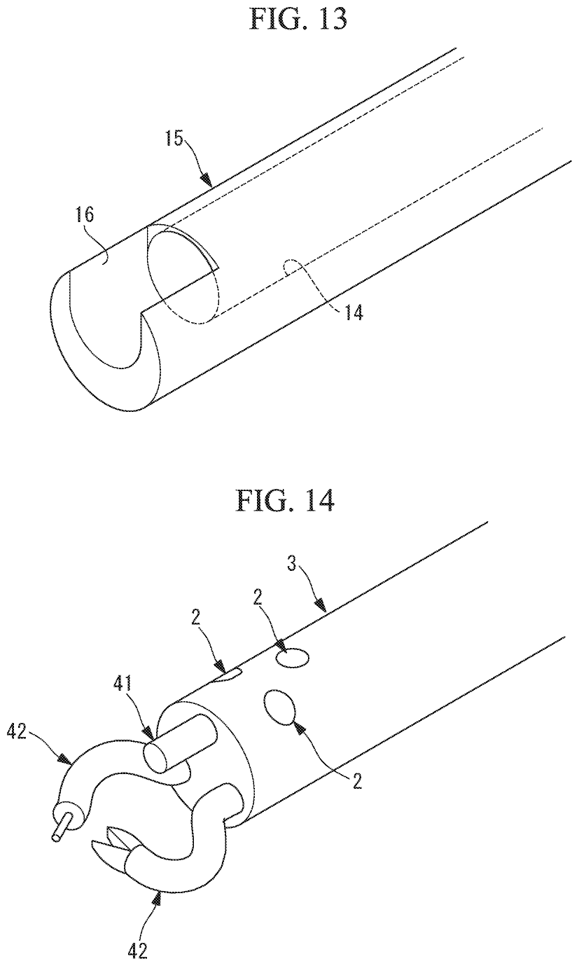

In addition, as shown in FIG. 13, a tube-shaped attachment 15 that has an eccentric inner hole 14 over nearly the entire length thereof may be employed. By doing so, it is possible to further enhance the torsional rigidity of the attachment 15. In FIG. 13, reference sign 16 is a notch that facilitates the introduction of the medical device 4 into the third lumen 8 when the attachment 15 is set at the second phase.

In addition, in this embodiment, although the two grooves 10 and the two protrusions 13 respectively engage with each other, alternatively, a single protrusion 13 may be provided. In addition, although the grooves 10 are provided at the two locations at 180°-intervals in the circumferential direction, three or more grooves 10 may be provided at predetermined intervals.

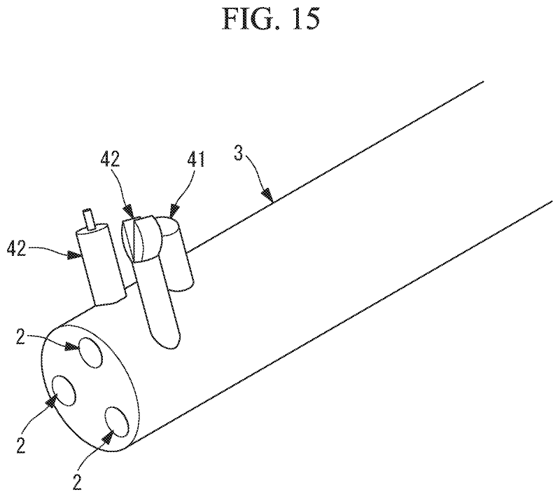

In addition, although the embodiment has been described in terms of an example in which the single lumen 2 is provided, alternatively, as shown in FIGS. 14 and 15, three lumens 2 may be provided, and one imaging catheter 41 and two treatment tools 42 may be employed as the medical devices.

By doing so, it is possible to easily switch between a first state in which the two treatment tools 42 are made to protrude at appropriate positions in the viewing field of the imaging catheter 41 protruded from the distal-end surface of the overtube 3, as shown in FIG. 14, and a second state in which the two treatment tools 42 are made to protrude at appropriate positions in the viewing field of the imaging catheter 41 protruded from the outer circumferential surface of the overtube 3, as shown in FIG. 15. In this case, because the protruding directions of the imaging catheter 41 and the treatment tools 42 are uniquely determined by the second lumen 7 or the third lumen 8, there is an advantage in that it is not necessary to perform fine adjustment of the protruding positions.

In addition, although the lumen from which the medical device 4 is made to protrude is switched between the lumens 7 and 8 by rotating the attachment 5 about the center axis of the first lumen 6 by 180°, alternatively, the switch may be made by means of a rotation by an arbitrary angle other than 180°.

An aspect of the present invention is a medical system including: an overtube that has a first lumen that extends in a longitudinal direction toward a distal end from a proximal end, a second lumen that branches off from the distal end of the first lumen and opens at a distal-end surface, and that is in communication with the first lumen via a first entrance, and a third lumen that opens at an outer circumferential surface and that is in communication with the first lumen via a second entrance; and an elongated attachment that is inserted, together with an elongated medical device, into the first lumen of the overtube from the proximal-end side, and that has a lateral cross-sectional shape that makes it possible to selectively blocks at least a portion of either the first entrance or the second entrance as a result of the position thereof about a center axis of the first lumen being changed and to guide the medical device to the other entrance.

With this aspect, the medical device is inserted, together with the attachment, into the first lumen from the proximal-end side of the overtube, at least a portion of the first entrance to the second lumen is blocked with the attachment, and thus, it is possible to guide the medical device into the third lumen. In addition, as a result of changing the position of the attachment about the center axis of the first lumen in the first lumen, at least a portion of the second entrance to the third lumen is blocked with the attachment, and thus, it is possible to guide the medical device into the second lumen.

Specifically, by merely changing the position of the attachment about the center axis of the first lumen by operating the elongated attachment on the proximal-end side of the overtube, it is possible to insert the medical device into either the second lumen or the third lumen. Therefore, it is not necessary to provide a raising base in the overtube in a pivotable manner, or to handle a wire for pivoting the raising base, and it is possible, with a simple structure, to selectively make a medical device protrude either forward with respect to a longitudinal direction of an overtube or in a direction that intersects the longitudinal direction.

The above-described aspect may include, at a position at which the second lumen and the third lumen branch off from the first lumen, an abutting surface against which a distal end of the attachment is abutted.

By doing so, as a result of abutting the distal end of the attachment, which is inserted from the proximal end of the first lumen, against the abutting surface provided at the position at which the second lumen and the third lumen branch off from the first lumen, the attachment is disposed in the state in which the attachment is positioned with respect to the overtube, and thus, it is possible to reliably block at least a portion of the first entrance to the second lumen or the second entrance to the third lumen and to reliably guide the medical device into the other lumen.

In addition with the above-described aspect, the position of a distal-end edge of the second entrance and the position of the abutting surface may be aligned in the longitudinal direction.

In addition, with the above-described aspect, the attachment may have, at least in a distal-end portion in the longitudinal direction, a substantially arc-like lateral cross-sectional shape that allows insertion into a gap between an inner surface of the first lumen and an outer surface of the medical device in a state in which the medical device is biased in a radial direction in the first lumen.

By doing so, it is possible to easily switch the position of the distal end of the medical device in the first lumen to the radial direction by changing the position of the distal-end portion of the attachment, which has a substantially arc-like lateral cross-sectional shape, in the first lumen by rotating the attachment about the center axis of the first lumen.

In addition, with the above-described aspect, the attachment may be formed in a tube shape having an inner hole into which the medical device can be inserted, and, at least in a distal-end portion in the longitudinal direction, the inner hole is eccentrically disposed in a radial direction with respect to a substantially circular external shape.

By doing so, it is possible to easily switch the position of the distal end of the medical device in the first lumen to the radial direction by changing the position of the inner hole provided at the distal end of the attachment by changing the position thereof in the first lumen by rotating the attachment about the center axis of the first lumen.

In addition, with the above-described aspect, the overtube may include two or more slits that extend along the first lumen in the longitudinal direction from the opening at the proximal end thereof, that extend radially outward from the inner surface of the first lumen, and that are disposed with spacings therebetween in a circumferential direction, and the attachment may include protrusions that extend radially outward from an outer circumferential surface of the attachment and that are capable of being inserted into the slits.

By doing so, it is possible to maintain the phase of the attachment about the center axis of the first lumen in a predetermined state by inserting the attachment into the first lumen and by inserting the protrusion of the attachment into any one of the slits provided in the inner surface of the first lumen. In addition, it is possible to maintain the phase of the attachment about the center axis of the first lumen in another state by inserting the protrusion of the attachment into another slit.

Specifically, it is possible to maintain the respective states by switching between a phase at which the first entrance to the second lumen is blocked with the attachment and a phase at which the second entrance to the third lumen is blocked with the attachment by merely changing the slit into which the protrusion is inserted.

### REFERENCE SIGNS LIST

1 medical system
3 overtube
4 medical device
5, 15 attachment
6 first lumen
7 second lumen
7a entrance (first entrance)
8 third lumen
8a entrance (second entrance)
9 abutting surface
10 groove (slit)
11, 14 inner hole
12 distal-end portion
13 protrusion
41 imaging catheter (medical device)
42 treatment tool (medical device)

1. A medical system comprising:
an overtube that has a first lumen that extends in a longitudinal direction toward a distal end from a proximal end, a second lumen that branches off from the distal end of the first lumen and opens at a distal-end surface, and that is in communication with the first lumen via a first entrance, and a third lumen that opens at an outer circumferential surface and that is in communication with the first lumen via a second entrance; and
an attachment that is configured to be inserted into the first lumen of the overtube, and that has a lateral cross-sectional shape that makes it possible to selectively block at least a portion of either the first entrance or the second entrance and to guide a medical device to the other entrance.

2. The medical system according to claim 1, further comprising, at a position at which the second lumen and the third lumen branch off from the first lumen:
an abutting surface against which a distal end of the attachment is abutted.

3. The medical system according to claim 2, wherein the position of a distal-end edge of the second entrance and the position of the abutting surface are aligned in the longitudinal direction.

4. The medical system according to claim 1, wherein the attachment has, at least in a distal-end portion in the longitudinal direction, a substantially arc-like lateral cross-sectional shape that allows insertion into a gap between an inner surface of the first lumen and an outer surface of the medical device in a state in which the medical device is biased in a radial direction in the first lumen.

5. The medical system according to claim 1, wherein the attachment is formed in a tube shape having an inner hole into which the medical device can be inserted, and, at least in a distal-end portion in the longitudinal direction, the inner hole is eccentrically disposed in a radial direction with respect to a substantially circular external shape.

6. The medical system according to claim 1,
wherein the overtube comprises two or more slits that extend along the first lumen in the longitudinal direction from the opening at the proximal end thereof, that extend radially outward from the inner surface of the first lumen, and that are disposed with spacings therebetween in a circumferential direction, and
the attachment comprises protrusions that extend radially outward from an outer circumferential surface of the attachment and that are capable of being inserted into the slits.

7. A medical system comprising:
a tubular body that has a first lumen that extends in a longitudinal direction toward a distal end from a proximal end, a second lumen that branches off from the distal end of the first lumen and opens at a distal-end surface, and that is in communication with the first lumen via a first entrance, and a third lumen that opens at an outer circumferential surface and that is in communication with the first lumen via a second entrance; and
an attachment that is configured to be inserted into the first lumen of the tubular body, and that has a lateral cross-sectional shape that makes it possible to selectively block at least a portion of either the first entrance or the second entrance and to guide a medical device to the other entrance.

8. A medical system comprising:
a tubular body that has a first lumen that extends in a longitudinal direction toward a distal end from a proximal end, a second lumen that branches off from the distal end of the first lumen and opens at a distal-end surface, and that is in communication with the first lumen via a first entrance, and a third lumen that opens at an outer circumferential surface and that is in communication with the first lumen via a second entrance; and
an attachment that is configured to be attached to and detached from the first lumen, wherein
the attachment includes a surface at a distal end portion thereof, the surface being inclined along a longitudinal axis, and
the surface is configured to be inclined toward the first entrance when at least a portion of the second entrance is blocked by the distal end portion of the attachment in a state in which the attachment is attached to the first lumen.
